(19) United States
(12) Patent Application Publication
(10) Pub. No.: US 2022/0061683 A1
(43) Pub. Date: Mar. 3, 2022

(54) BLOOD PRESSURE MEASURING APPARATUS, MODEL SETTING DEVICE, AND BLOOD PRESSURE MEASURING METHOD

(71) Applicant: SHARP KABUSHIKI KAISHA, Sakai City, Osaka (JP)

(72) Inventors: YOSHIHISA ADACHI, Sakai City, Osaka (JP); RIEKO OGAWA, Sakai City, Osaka (JP); YUKI EDO, Sakai City, Osaka (JP); RYOTA TOMIZAWA, Sakai City, Osaka (JP)

(21) Appl. No.: 17/420,330

(22) PCT Filed: Dec. 20, 2019
(86) PCT No.: PCT/JP2019/050082

§ 371 Date: Jul. 1, 2021

(30) Foreign Application Priority Data
Jan. 9, 2019 (JP) 2019-001951

(51) Int. Cl. A61B 5/021; A61B 5/0205; A61B 5/11

(52) U.S. Cl. CPC A61B 5/02108; A61B 5/1122; A61B 5/0205

(57) ABSTRACT

A blood-pressure measuring apparatus includes: a pulse-wave acquiring unit configured to acquire a pulse wave in a predetermined region on a body surface of a living body; a body-movement detector configured to detect a direction of movement of the predetermined region; a body-movement classifier configured to classify the direction of movement; and a pulse-wave-parameter calculator configured to calculate a plurality of pulse wave parameters based on the pulse wave. The blood-pressure measuring apparatus is communicably connected to a model storage. The model storage stores in advance a blood-pressure estimation model for estimating a first blood pressure in response to a resultant classification of the direction of movement. The blood-pressure measuring apparatus further comprises a first-blood-pressure measuring unit configured to calculate, by using the blood-pressure estimation model, the first blood pressure based on the plurality of pulse wave parameters.

### TECHNICAL FIELD

An aspect of the present disclosure relates to a blood-pressure measuring apparatus that measures the blood pressure of a living body on the basis of the pulse wave of the living body. The present application claims priority from Japanese Patent Application No. 2019-1951, filed on Jan. 9, 2019, the content of which is hereby incorporated by reference into this application.

### BACKGROUND ART

Various techniques have been recently proposed for measuring information about a living body (subject). For example, Patent Literature 1 discloses a technique for measuring, with high accuracy, a predetermined kind of information (e.g., pulse rate) about a living body or subject on the basis of an image (camera image) on which the subject's face is appearing. To be specific, the technique in Patent Literature 1 aims to measure the pulse rate with high accuracy even when the subject's face moves.

### CITATION LIST

### Patent Literature

Patent Literature 1: Japanese Patent Application Laid-Open No. 2017-93760

### SUMMARY OF INVENTION

### Technical Problem

However, Patent Literature 1 is silent about a specific method for measuring the blood pressure of a living body (another example of living-body information) with high accuracy. It is an object of one aspect of the present disclosure to measure the blood pressure of a living body more accurately than before.

### Solution to Problem

To solve the above problem, a blood-pressure measuring apparatus according to one aspect of the present disclosure measures a first blood pressure of a living body on the basis of the pulse wave of the living body. The blood-pressure measuring apparatus includes the following: a pulse-wave acquiring unit that acquires the pulse wave in a predetermined region on the body surface of the living body; a body-movement detector that detects a direction of movement of the predetermined region; a body-movement classifier that classifies the direction of movement; and a pulse-wave-parameter calculator that calculates a plurality of pulse wave parameters based on the pulse wave. The blood-pressure measuring apparatus is communicably connected to a model storage. The model storage stores in advance (i) a plurality of blood-pressure estimation models for estimating the first blood pressure in response to a resultant classification of the direction of movement, and (ii) resultant evaluations of the plurality of individual blood-pressure estimation models based on the resultant classification of the direction of movement. The blood-pressure measuring apparatus further includes the following: a model selector that selects a measurement model for calculating the first blood pressure from among the plurality of blood-pressure estimation models in response to the resultant evaluations of the plurality of individual blood-pressure estimation models; and a first-blood-pressure measuring unit that calculates, by using the measurement model, the first blood pressure based on the plurality of pulse wave parameters.

To solve the above problem, a model setting device according to one aspect of the present disclosure is communicably connected to a blood-pressure measuring apparatus that measures a first blood pressure of a living body on the basis of the pulse wave of the living body, the model setting device includes the following: a second-blood-pressure measuring unit that measures a second blood pressure of the living body; a pulse-wave acquiring unit that acquires the pulse wave in a predetermined region on the body surface of the living body; a body-movement detector that detects a direction of movement of the predetermined region; a body-movement classifier that classifies the direction of movement; and a pulse-wave-parameter calculator that calculates a plurality of pulse wave parameters based on the pulse wave. The model setting device is communicably connected to a model storage. The model setting device further includes a model creating unit that creates, based on the plurality of pulse wave parameters and the second blood pressure, a plurality of blood-pressure estimation models for estimating the first blood pressure in response to a resultant classification of the direction of movement. The model creating unit also stores the plurality of blood-pressure estimation models in the model storage. The model setting device further includes a model evaluating unit that individually evaluates the plurality of blood-pressure estimation models stored in the model storage in response to the resultant classification of the direction of movement. The model evaluating unit also stores resultant evaluations of the plurality of individual blood-pressure estimation models in the model storage.

To solve the above problem, a blood-pressure measuring method using a blood-pressure measuring apparatus that measures a first blood pressure of a living body on the basis of the pulse wave of the living body. The blood-pressure measuring method includes the following steps: acquiring the pulse wave in a predetermined region on the body surface of the living body; detecting a direction of movement of the predetermined region; classifying the direction of movement; and calculating a plurality of pulse wave parameters based on the pulse wave. The blood-pressure measuring apparatus is communicably connected to a model storage. The model storage stores in advance (i) a plurality of blood-pressure estimation models for estimating the first blood pressure in response to a resultant classification of the direction of movement, and (ii) resultant evaluations of the plurality of individual blood-pressure estimation models based on the resultant classification of the direction of movement. The blood-pressure measuring method further includes the following steps: selecting a measurement model for calculating the first blood pressure from among the plurality of blood-pressure estimation models in response to the resultant evaluations of the plurality of individual blood-pressure estimation models; and calculating, by using the measurement model, the first blood pressure based on the plurality of pulse wave parameters.

### Advantageous Effect of Invention

The blood-pressure measuring apparatus according to the aspect can measure the blood pressure of a living body more accurately than before. The blood-pressure measuring method according to the aspect offers a similar effect. The model setting apparatus according to the aspect of the present disclosure offers a similar effect.

### BRIEF DESCRIPTION OF DRAWINGS

FIG. 1 is a functional block diagram illustrating the configuration of main components of a blood-pressure measuring apparatus according to a first embodiment.

FIG. 2 illustrates an example process step performed in a facial-image divider.

FIG. 3 illustrates another example process step performed in the facial-image divider.

FIG. 4 illustrates example extraction of a measurement model candidate.

FIG. 5 is an example face-direction template.

FIG. 6 is a flowchart illustrating, by way of example, how the blood-pressure measuring apparatus in FIG. 1 creates a measurement model.

FIG. 7 is a flowchart illustrating, by way of example, how the blood-pressure measuring apparatus in FIG. 1 measurements blood pressure.

### DESCRIPTION OF EMBODIMENTS

### First Embodiment

The following describes a blood-pressure measuring apparatus 1 according to a first embodiment. For convenience, components of the same functions as those described in the first embodiment will be denoted by the same sings and will not be elaborated upon in the subsequent embodiments. Descriptions similar to publicly known techniques will be omitted as necessary. Configurations in the individual drawings are illustrative for convenience in description; so are numeric values in the Description.

Outline of Blood-Pressure Measuring Apparatus 1

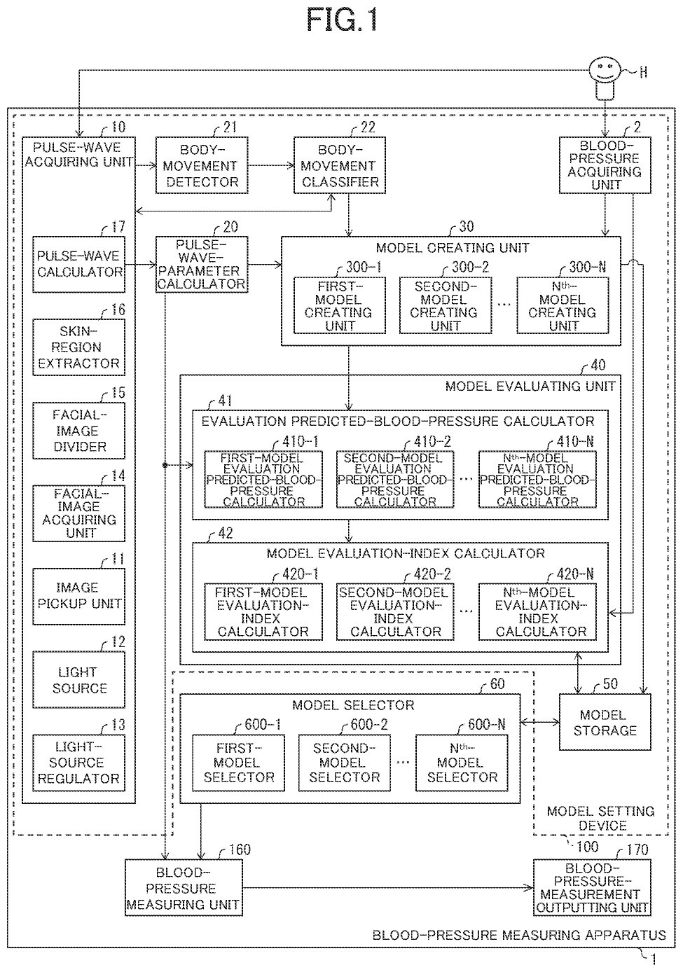

FIG. 1 is a functional block diagram illustrating the configuration of main components of a blood-pressure measuring apparatus 1. The blood-pressure measuring apparatus 1 measures the blood pressure of a subject H (hereinafter merely referred to as blood pressure) on the basis of the pulse wave of the subject (living body) H. To be specific, the blood-pressure measuring apparatus 1 measures the blood pressure using a blood-pressure measurement model that is set in a model setting device 100 (hereinafter also merely referred to as a “measurement model”), which will be described below. It is noted that a blood-pressure estimation model, described later on, is also merely referred to as an estimation model in the Description. It is also noted that a measurement model and an estimation model are also merely referred to as a model generically.

The following describes the blood-pressure measuring apparatus 1, which is a contactless blood-pressure measuring apparatus (a blood-pressure measuring apparatus capable of measuring the blood pressure without contacting the subject H). The first embodiment describes an instance where the subject H is a human. The blood-pressure measuring apparatus 1 measures the blood pressure by using a predetermined region on the body surface of the subject H as a region of interest (ROI). The following describes an instance where an ROI is the face. In the Description, the face of the subject H is also merely referred to as a face. This holds true for the other indications.

The blood-pressure measuring apparatus 1 includes the model setting device 100, a model selector 60, a blood-pressure measuring unit 160 (first-blood-pressure measuring unit), and a blood-pressure-measurement outputting unit 170. The model setting device 100 includes a blood-pressure acquiring unit 2 (second-blood-pressure measuring unit), a pulse-wave acquiring unit 10, a pulse-wave-parameter calculator 20, a body-movement detector 21, a body-movement classifier 22, a model creating unit 30, a model evaluating unit 40, and a model storage 50.

FIG. 1 illustrates an instance where the model setting device 100 is placed inside the blood-pressure measuring apparatus 1. The model setting device 100 can be placed outside the blood-pressure measuring apparatus 1 (see a second embodiment, which will be described later on).

The blood-pressure acquiring unit 2 measures the blood pressure of the subject H. The blood-pressure acquiring unit 2 is a contact blood-pressure monitor (e.g., a cuff blood-pressure monitor). The blood pressure (hereinafter, BPm) measured by the blood-pressure acquiring unit 2 is used as test data (training data) in the model setting device 100. That is, the BPm is used for setting a measurement model in the model selector 60. The BPm is also used for setting a plurality of estimation models in the model creating unit 30.

The blood-pressure acquiring unit 2 outputs the BPm to the model creating unit 30 and model evaluating unit 40 (more specifically; to a model evaluation-index calculator 42, which will be described below). Here, the final resultant blood pressure (P, which will be described later on) measured by the blood-pressure measuring apparatus 1 is also referred to as a first blood pressure. In addition, the BPm is also referred to as a second blood pressure in order to distinguish the BPm from the first blood pressure. As described later on, the P is measured (calculated) by the blood-pressure measuring unit 160. The BPm can be thus also expressed as training data for the blood-pressure measuring apparatus 1 to measure the P.

Pulse-Wave Acquiring Unit 10

The pulse-wave acquiring unit 10 acquires the pulse wave in a ROI (e.g., the face). The pulse-wave acquiring unit 10 includes an image pickup unit 11, a light source 12, a light-source regulator 13, a facial-image acquiring unit 14, a facial-image divider 15, a skin-region extractor 16, and a pulse-wave calculator 17.

The image pickup unit 11 is a camera that includes an image sensor and a lens. The image sensor may be a complementary metal-oxide semiconductor (CMOS) image sensor or a charge-coupled device (CCD) image sensor, for instance. The image pickup unit 11 takes an image of the subject 14 multiple times and outputs the resultant image of the subject H (hereinafter, referred to as a subject image) to the facial-image acquiring unit 14 at a predetermined frame rate (i.e., at predetermined time intervals). The frame rate is 300 fps (frames per second) for instance.

The image pickup unit 11 may include a publicly known color filter. This color filter preferably has optical properties suitable for observing fluctuations in blood volume. Suitable examples of the color filter include (i) a red-blue-green-cyan (RGBCy) color filter and (ii) a red-blue-green-infrared (RGBIR) color filter. Moreover, the image pickup unit 11 may be an RGB camera or an IR camera.

The light source 12 emits light to the subject H when the image pickup unit 11 takes an image of the subject H. The light-source regulator 13 regulates the light source 12. For instance, the light-source regulator 13 preferably regulates the light source in such a manner that the time of pulse wave propagation (an example pulse-wave parameter) between regions used in a measurement model selected by the model selector 60 can be calculated accurately.

To be specific, the light-source regulator 13 regulates the light source 12 in such a manner that a pulse wave of predetermined signal quality in a pertinent region can be detected. Herein, a pulse wave of predetermined signal quality refers to a pulse wave having a high single-to-noise ratio (SNR). To be more specific, the light-source regulator 13 regulates at least one of (i) the amount of light from the light source 12, (ii) the light spectrum of the light source 12, and (iii) the angle of irradiation to the skin of the subject H.

The pulse-wave acquiring unit 10 does not necessarily have to include the light source 12 and light-source regulator 13. Without the light source 12 and light-source regulator 13, the image pickup unit 11 may use only ambient light to take an image of the subject H.

The facial-image acquiring unit 14 extracts a facial region of the subject H from a subject image taken by the image pickup unit 11. The facial-image acquiring unit 14 acquires an image that has undergone facial-region extraction, as a facial image (an image on which the face of the subject H is appearing), For instance, the facial-image acquiring unit 14 may perform face tracking on a moving image (a moving image consisting of a plurality of subject images) on which the subject is appearing, to extract a facial region for each predetermined frame of the moving image.

Nevertheless, the facial-image acquiring unit 14 can extract a facial region without necessarily performing face tracking. For instance, the image pickup unit 11 may take a subject image, (i) with the face of the subject H placed within a predetermined frame, and (ii) with the face and image pickup unit 11 fixed. This configuration, which can prevent facial blurring in the subject image, requires no face tracking.

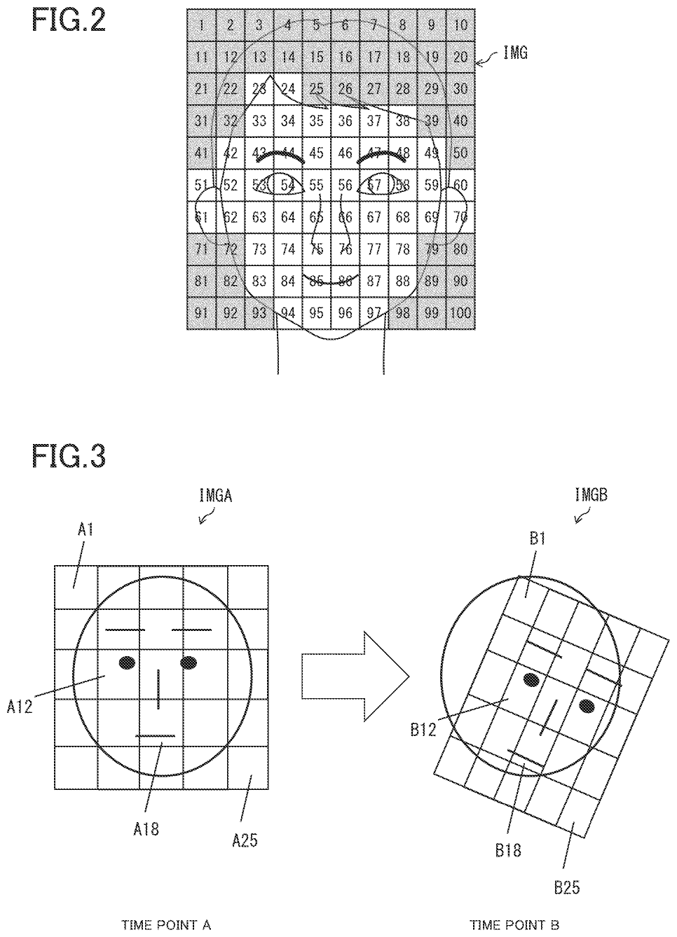

The facial-image divider 15 divides a facial image extracted by the facial-image acquiring unit 14 into a plurality of regions (partial regions). For convenience in description, a facial image is hereinafter referred to as an IMG. FIG. 2 illustrates an example process step performed in the facial-image divider 15. FIG. 2 illustrates an example IMG that has been divided by the facial-image divider 15. The IMG in FIG. 2 is an example facial image on which a front-facing face is appearing.

In the example of FIG. 2, the facial-image divider 15 divides the IMG into tenth vertically and horizontally into tenth equally in both directions). That is, the facial-image divider 15 divides the IMG into 100 partial regions (Partial Regions 1 to 100). Herein, how to divide the IMG by the facial-image divider 15 is not limited to the example in FIG. 2. For instance, the sizes of the partial regions do not necessarily have to be the same.

Prior to the description of the other components of the pulse-wave acquiring unit 10, the following describes the operation of the body-movement detector 21. The body-movement detector 21 detects a body movement of the subject H. To be specific, the body-movement detector 21 detects a movement of an ROI (e.g., face). To be more specific, the body-movement detector 21 detects a direction of movement of the ROI. For instance, the body-movement detector 21 uses the result of face tracking, performed by the facial-image acquiring unit 14, to detect the amount of movement of each facial feature point (e.g., the eyes, nose, mouth, and contour) in a moving image.

To be specific, the body-movement detector 21 detects the amount of positional change (the amount of movement) in each feature point for each predetermined frame. That is, the body-movement detector 21 detects, for each predetermined frame, where and how much each feature point has moved. The body-movement detector 21 further detects a direction of facial movement on the basis of the foregoing movement amount. The body-movement detector 21 further detects the orientation of the face as of now on the basis of the foregoing movement amount.

As described later on, the model setting device 100 sets a plurality of kinds of predetermined patterns in advance with regard to the direction of movement (e.g., face orientation) of an ROI. As detailed below, the body-movement classifier 22 identifies which of these predetermined patterns the face orientation, detected by the body-movement detector 21, belongs to (falls under).

FIG. 3 illustrates another example process step performed in the facial-image divider 15. The facial-image divider 15 can divide an IMG further on the basis of the result of pattern classification, performed by the body-movement classifier 22. IMGA in FIG. 3 is another example facial image on which a front-facing face is appearing. The body-movement classifier 22 determines that the orientation of the thee in the IMGA falls under Pattern 1 shown in FIG. 5, which will be described later on.

In the example of FIG. 3, how to divide the IMGA is similar to that in the example of FIG. 2. In the example of FIG. 3, the IMGA is divided into 25 partial regions (partial regions A1 to A25) for convenience in description. For easy illustration, only some of Partial Regions A1 to A25 are shown with their reference numbers in FIG. 3. This holds true for Partial Regions B1 to B25 (the partial regions of IMGB), which is described below.

The IMGB in FIG. 3 is an example facial image on which a face facing lower right is appearing. Hereinafter, when the IMGA has been taken is referred to as Time Point A, and when the IMGB has been taken is referred to as Time Point B. Time Point B is posterior to Time Point A in this example. The body-movement classifier 22 determines that the orientation of the face in IMG2 falls under Pattern 7 shown in FIG. 5.

The facial-image divider 15 divides a facial image of one pattern (e.g., IMGB, which is the facial image of Pattern 7) on the basis of the result of division performed on a facial image of another pattern (e.g., IMGA, which is the facial image of Pattern 1). To be specific, the facial-image divider 15 divides the IMGB in such a manner that the partial regions (B1 to B25) of IMGB correspond to the respective partial regions of the IMGA. In the example of FIG. 3, B1 corresponds to A1, and B25 corresponds to A25.

Such dividing of a facial image enables the substantially same part to be shown between (i) a certain partial region at Time Point A (before a body movement in the subject H) and (ii) a partial region at Time Point B (after the body movement in the subject H) corresponding to the certain partial region. In the example of FIG. 3, A12 and B12 are partial regions on which one of the eyes (e.g., left eye) of the subject H is appearing. Further, A18 and B18 are partial regions on which the mouth of the subject H is appearing.

The skin-region extractor 16 extracts skin regions (regions on which at least part of the skin is appearing) from the partial regions. Each skin region can be expressed as a region in which the skin is not completely covered with an object (e.g., hair). In the example of FIG. 2, the skin regions are regions not shaded among the partial regions. In the example of FIG. 2, the skin-region extractor 16 extract 52 skin regions from among the 100 partial regions.

The pulse-wave calculator 17 calculates the pulse wave (more strictly; a pulse wave signal) for each of the skin regions extracted by the skin-region extractor 16. How to calculate the pulse wave in the pulse-wave calculator 17 may use a publicly known method (e.g., a method using independent component analysis). The pulse-wave calculator 17 supplies the resultant pulse wave to the pulse-wave-parameter calculator 20.

The pulse-wave-parameter calculator 20 calculates a pulse wave parameter on the basis of the pulse wave in each skin region acquired from the pulse-wave calculator 17. A pulse wave parameter in the Description generically refers to an explanatory variable (also called an independent variable) that is used in blood pressure measurement (calculation) based on a measurement model.

The first embodiment addresses an instance where a pulse transit time (PTT) between skin regions is used as a pulse wave parameter. In this case, the pulse-wave-parameter calculator 20 uses a publicly known method to calculate a PTT on the basis of the pulse wave in each skin region. It is noted that the PTT between Region A (any one skin region) and Region B (another skin region different from Region B) is also expressed as PTT (A-B). For instance, the PTT between the regions 23 and 24 in FIG. 2 is expressed as PTT (23-24).

In the example of FIG. 2, the pulse-wave-parameter calculator 20 selects a combination of any two skin regions from among the 52 skin regions. That is, the pulse-wave-parameter calculator 20 selects ₅₂C₂=1326 combinations in total. The pulse-wave-parameter calculator 20 then calculates a PTT for each combination. The pulse-wave-parameter calculator 20 thus calculates 1326 combinations of PTTs, that is, PTT (23-24) to PTT (96-97). The pulse-wave-parameter calculator 20 supplies each resultant PTT (pulse wave parameter) to the model creating unit 30, an evaluation predicted-blood-pressure calculator 41, and the blood-pressure measuring unit 160.

Model Creating Unit 30

The model creating unit 30 creates a blood-pressure estimation model (estimation model). An estimation model refers to a calculation model for estimating the blood pressure (P) of the subject H. To be specific, the model creating unit 30 creates the estimation model by using, as test data, (i) a pulse wave parameter (PTT) calculated by the pulse-wave-parameter calculator 20 and (ii) the blood pressure (BPm) of the subject H acquired by the blood-pressure acquiring unit 2.

As illustrated in FIG. 1, the model creating unit 30 has a first-model creating unit 300-1, a second-model creating unit 300-2 . . . , and an N^{th}-model creating unit 300-N. N denotes the number of classification patterns that are set in advance regarding face orientation, N is any integer equal to or greater than two. The k^{th}-model creating unit 300-k creates an estimation model based on Pattern k. Herein, k is an integer satisfying 1≤k≤N. As described, the model creating unit 30 can create an estimation model based on each pattern of the face orientation.

In the Description, the first-model creating unit 300-1 to the N^{th}-model creating unit 300-N are also generically referred to as the model creating unit 30 for convenience. The description about the model creating unit 30 is applied to any k^{th}-model creating unit 300-k. Likewise, in the Description, a first-model evaluation predicted-blood-pressure calculator 410-1 to an N^{th}-model evaluation predicted-blood-pressure calculator 410-N, all described later on, are also generically referred to as the evaluation predicted-blood-pressure calculator 41. In addition, a first-model evaluation-index calculator 420-1 to an N^{th}-model evaluation-index calculator 420-N, all described later on, are also generically referred to as the model evaluation-index calculator 42. In addition, a first-model selector 600-1 to an N^{th}-model selector 600-N, all described later on, are also generically referred to as the model selector 60.

Example Method for Creating Estimation Model

Velocity v, the speed at which a pulse wave propagates through a blood vessel, is expressed with the Moens-Korteweg equation as follows.

$$\begin{matrix}[ Expression 1 ] & \\ v = \sqrt{\frac{Ea}{2 R ρ}} & ( 1 )\end{matrix}$$

In Expression (1), E denotes the Young's modulus of the blood vessel, a denotes the wall pressure of the blood vessel, R denotes the diameter of the blood vessel, and ρ denotes blood density.

It is known that the Young's modulus E of a blood vessel exponentially varies with respect to blood pressure P. Accordingly, F is expressed as follows, where E0 is the Young's modulus of a blood vessel when P =0 is satisfied.

[Expression 2]

E=E₀e^{γ}^{^{P}} (2)

Here, γ is a constant that depends on the blood vessel.

The length L of the blood vessel pathway is expressed as follow.

[Expression 3]

L=vT (3)

Here, T denotes a pulse transit time (PTT), and L denotes the length of the blood vessel pathway.

The following expression is derived from Expressions (1) to (3).

$$\begin{matrix}[ Expression 4 ] & \\ P = \frac{1}{γ} ( ln \frac{1}{{T}^{2}} + ln \frac{2 {R ρ L}^{2}}{{E}_{0} a} ) & ( 4 )\end{matrix}$$

As shown in Expression (4), T and P establish a correlation when L is constant.

The model creating unit 30 thus creates a plurality of estimation models for P using a PTT calculated by the pulse-wave-parameter calculator 20.

Firstly, the model creating unit 30 creates an estimation model M1 of Complexity Level 1. In the Description, a complexity level refers to the number of explanatory variables in an estimation model (e.g., the number of PTTs used in an estimation model). The estimation model M1 uses one PTT as an explanatory variable in the following instance.

In the following description, one PTT calculated by the pulse-wave-parameter calculator 20 is expressed as PTT1. The PTT1 is the PTT between any two skin regions. The model creating unit 30 performs regression analysis on the PTT1 and BPm through the method of least squares. The model creating unit 30 creates the estimation model M1 as a result of the regression analysis. Each PTT calculated by the pulse-wave-parameter calculator 20 and the BPm acquired by the blood-pressure acquiring unit 2 are both example test data.

Reference is made to an instance where the estimation model M1 is a linear model (a calculation model expressed by a linear function expressed as Expression (5) below.

BP1=α1×PTT1+α2 (5)

In Expression (5), the BP1 denotes a predicted blood pressure, and each of the α1 and α2 is a constant. The model creating unit 30 in this case performs regression analysis to calculate the α1 and α2 (that is, create the estimation model M1).

Hereinafter, the respective PTTs of the 1326 combinations in the example of FIG. 2 are referred to as PTT1-1 to PTT1-1326 for convenience. The model creating unit 30 uses the PTT1-1 to PTT1-1326 to create as many estimation models M1 as these PTTs. The respective estimation models M1 are referred to as M1-1 to M1-1326 for convenience.

Secondly, the pulse-wave-parameter calculator 20 creates an estimation model M2 of Complexity Level 2. The estimation model M2 uses two PTTs as explanatory variables. In the following description, two mutually different PTTs calculated by the pulse-wave-parameter calculator 20 are expressed as PTT1 and PTT2.

The model creating unit 30 performs regression analysis on (i) the PTT1 and PTT2 and (ii) the BPm through the method of least squares. The model creating unit 30 creates an estimation model M2 as a result of the regression analysis.

Reference is made to an instance where the estimation model M2 is a linear model expressed as Expression (6) below.

BP2=β1×PTT1+β2×PTT2+β3 (6)

In Expression (6), the BP2 denotes a predicted blood pressure, and each of the β1 to and β3 is a constant. The model creating unit 30 in this case performs regression analysis to calculate the β1 to and β3.

In the example of FIG. 2, the model creating unit 30 uses the PTT1-1 to PTT1-1326 to create more estimation models M2 than these PTTs. There are 878475 combinations (i.e., ₁₃₂₆C₂ combinations) of the PTT1 and PTT2 in this example. The model creating unit 30 thus creates 878475 estimation models M2.

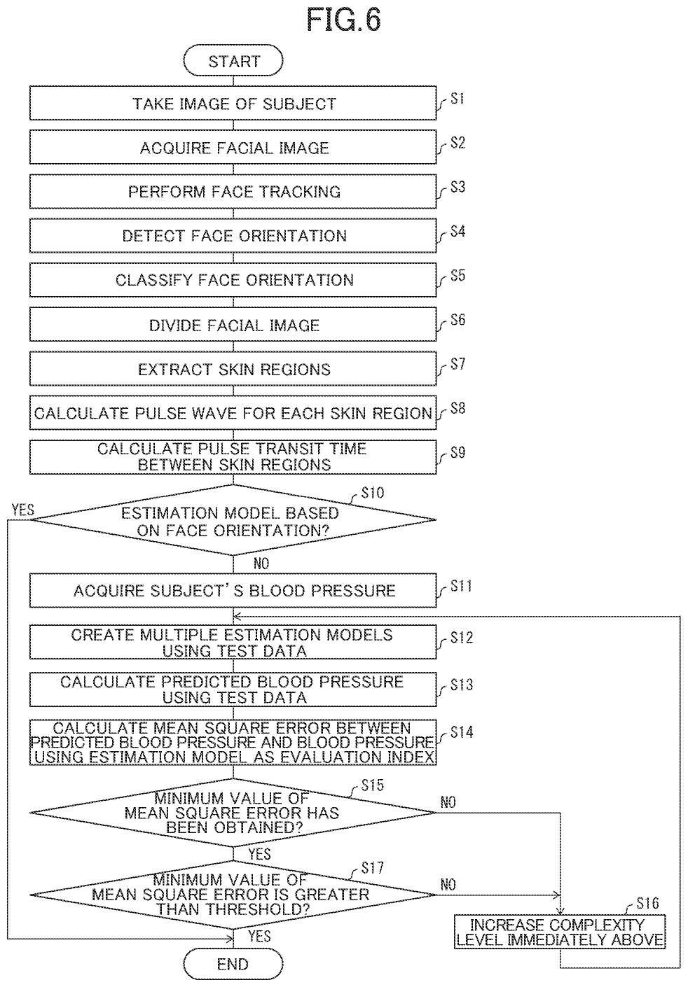

Likewise, the model creating unit 30 creates an estimation model M3 of Complexity Level 3, an estimation model M4 of Complexity Level 4 . . . , an estimation model Mz of Complexity Level z. Herein, z denotes the maximum of the complexity level. Further, the z depends on resultant calculations in the individual process steps in the flowchart of FIG. 6, which will be described later on. The model creating unit 30 supplies each resultant estimation model to the model evaluating unit 40 (to be more specific, to the evaluation predicted-blood-pressure calculator 41).

As described above, the first-model creating unit 300-1 creates an estimation model based on Pattern 1 (hereinafter, a first model); so do the second-model creating unit 300-2 to the N^{th}-model creating unit 300-N. That is, the k^{th}-model creating unit 300-k creates an estimation model based on Pattern k (hereinafter, a k^{th} model).

The model creating unit 30 creates the first to N^{th} models in this way. The first to N^{th} models are hereinafter also generically referred to as a model group. In the example of FIG. 1, the model creating unit 30 supplies the resultant model group to the model evaluating unit 40 and model storage 50.

Model Evaluating Unit 40

The model evaluating unit 40 evaluates each estimation model created by the model creating unit 30 and outputs the resultant evaluation. To be specific, the model evaluating unit 40 outputs a PI, described below, as the resultant evaluation. In the example of FIG. 1, the model evaluating unit 40 acquires the model group directly from the model creating unit 30. In some embodiments, the model evaluating unit 40 may acquire a model group created by the model creating unit 30 and stored in advance in the model storage 50.

The model evaluating unit 40 has the evaluation predicted-blood-pressure calculator 41 and the model evaluation-index calculator 42. The evaluation predicted-blood-pressure calculator 41 has the first-model evaluation predicted-blood-pressure calculator 410-1, the second model evaluation predicted-blood-pressure calculator 410-2 . . . , and the N^{th}-model evaluation predicted-blood-pressure calculator 410-N. The model evaluation-index calculator 42 has the first-model evaluation-index calculator 420-1, the second-model evaluation-index calculator . . . , and the N^{th}-model evaluation-index calculator 420-N.

The k^{th}-model evaluation predicted-blood-pressure calculator 410-k and the k^{th}-model evaluation-index calculator 420-k are functional units appropriate to the k^{th} model. The k^{th}-model evaluation predicted-blood-pressure calculator 410-k and the k^{th}-model evaluation-index calculator 420-k are also generically referred to as a k^{th}-model evaluating unit.

The evaluation predicted-blood-pressure calculator 41 calculates a predicted blood pressure (hereinafter, BPe) in an estimation model created by the model creating unit 30. To be specific, the evaluation predicted-blood-pressure calculator 41 calculates the BPe by applying (to be specific, substitutes), to the estimation model, a PTT calculated as test data by the pulse-wave-parameter calculator 20.

The model evaluation-index calculator 42 calculates the evaluation index (hereinafter, PI) of the estimation model. The model evaluation-index calculator 42 may calculate the PI on the basis of the BPe and BPm. For instance, the model evaluation-index calculator 42 calculates, as the PI, the mean square error (MSE) between the BPe and BPm. The model evaluation-index calculator 42 calculates the PI of each estimation model in ascending order of the complexity level of the estimation models. The model evaluation-index calculator 42 then supplies the resultant PI to the model storage 50.

As described above, the first-model evaluation predicted-blood-pressure calculator 410-1 calculates a predicted blood pressure in the first model (a predicted blood pressure for the first model); so do the second model-evaluation predicted-blood-pressure calculator 410-2 to the N^{th}-model evaluation predicted-blood-pressure calculator 410-N. That is, the k^{th}-model evaluation predicted-blood-pressure calculator 410-k calculates a predicted blood pressure fir the k^{th} model (hereinafter, BPek). The evaluation predicted-blood-pressure calculator 41 calculates BPe1 to BPeN in this way. Hereinafter, the BPe1 to BPeN are also generically referred to as a predicted-blood-pressure group. The evaluation predicted-blood-pressure calculator 41 supplies the resultant predicted-blood-pressure group to the model evaluation-index calculator 42.

Subsequently, the first-model evaluation-index calculator 420-1 calculates each PI in the first model (a first-model evaluation-index set); so do the second-model evaluation-index calculator 420-2 to the N^{th}-model evaluation-index calculator 420-N. That is, the k^{th}-model evaluation-index calculator 420-k calculates a k^{th}-model evaluation-index set (hereinafter, PIk). Hereinafter, the PI1 to PIk are also generically referred to as a group of evaluation-index sets. The model evaluation-index calculator 42 associates the resultant group of evaluation-index sets with the model group and supplies them to the model storage 50.

Model Storing Unit 50

The model storage 50 stores (retains) the model group created by the model creating unit 30. The model storage 50 also stores the group of evaluation-index sets calculated by the model evaluation-index calculator 42. The model storage 50 may be a publicly known storage.

Model Selecting Unit 60

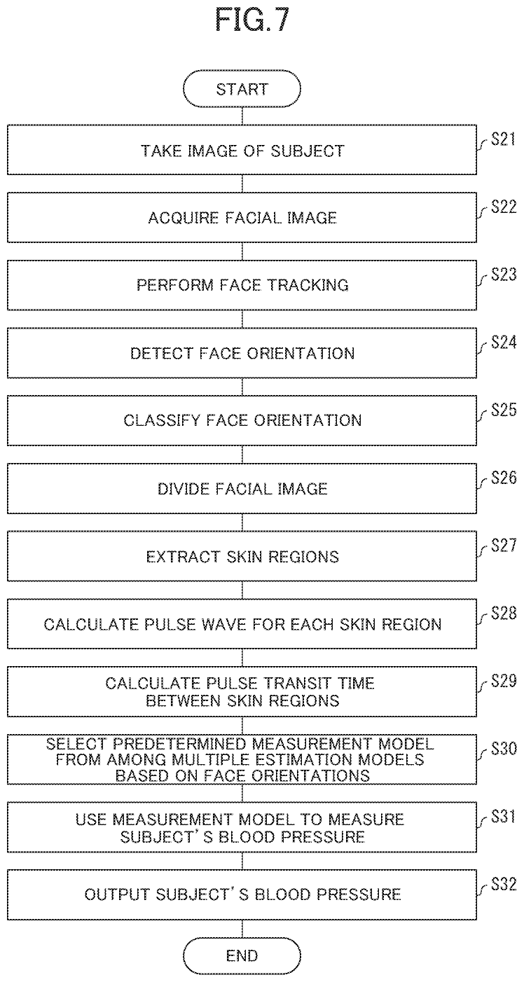

The model selector 60 has the first-model selector 600-1, the second model selector 600-2 . . . , and the N^{th}-model selector 600-N. The k^{th}-model selector 600-k is a functional unit appropriate to the k^{th} model. As illustrated in FIG. 7, which will be described later on, the model selector 60 operates for measuring (calculating) the blood pressure (P) in the blood-pressure measuring unit 160 after the model setting device 100 finish processing.

The model selector 60 selects at least one measurement model from among the plurality of estimation models stored in the model storage 50 on the basis of the resultant evaluations (i.e., each PI stored in the model storage 50) made by the model evaluating unit 40 (in more detail, the model evaluation-index calculator 42). A measurement model refers to a calculation model for measuring the blood pressure (P) in the blood-pressure measuring unit 160.

Firstly, the model selector 60 selects at least one measurement model candidate (model candidate) from among the estimation models. For instance, the model selector 60 extracts, as measurement model candidates, estimation models each having a PI (e.g., an MSE) equal to or less than a predetermined threshold from among the estimation models. The model selector 60 then selects at least one measurement model from among the measurement model candidates.

For instance, let the model selector 60 select one measurement model. Accordingly, the model selector 60 may select, as a measurement model, an estimation model having a PI that is the smallest of those of the measurement model candidates. Alternatively, the model selector 60 may select, as a measurement model, an estimation model having a complexity level that is the smallest of those of the measurement model candidates.

The model selector 60 may select a plurality of measurement models. For instance, the model selector 60 may select, as measurement models, measurement model candidates each having an SNR that is equal to or greater than a predetermined value in all partial regions used in the measurement model candidate from among a plurality of measurement model candidates.

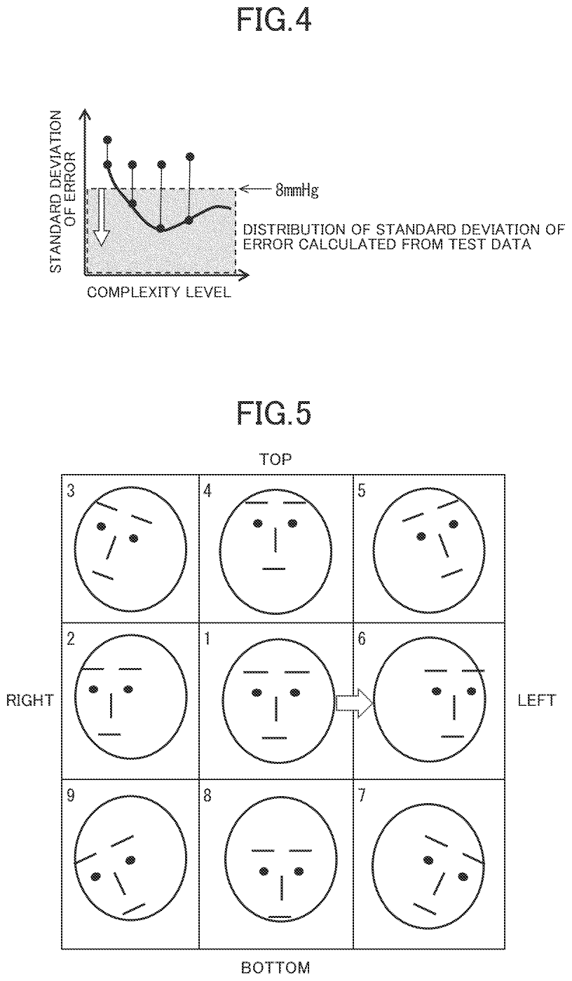

FIG. 4 illustrates example extraction of a measurement model candidate. In the example of FIG. 4, the standard deviation of an error in an estimation model is used as a (see a modification as well, which will be described later on). The model evaluation-index calculator 42 in this case calculates the standard deviation of the error between BPe (predicted blood pressure) and BPm (test data) as a P1. FIG. 4 is a graph showing the distribution of the PI (the standard deviation of the error) calculated by the model evaluation-index calculator 42.

In the example of FIG. 4, the predetermined threshold (blood pressure threshold) is set to be 8 mmHg. This value is set based on the standards of non-invasive blood pressure monitors. In this case, the model selector 60 extracts, as a measurement model candidate, an estimation model having a PI equal to or less than 8 mmgHg. In the example of FIG. 4, the model selector 60 extracts estimation models M2 to M4 from among estimation models M1 to M4 (Complexity Levels 1 to 4) as measurement model candidates.

As described above, the first-model selector 600-1 selects one of more measurement model candidates (intra-first-model measurement model candidates) from the first model. The first-model selector 600-1 then selects at least one measurement model (intra-first-model measurement model) from among the intra-first-model measurement model candidates. This holds true for the second model selector 600-2 to the N^{th}-model selector 600-N.

That is, the k^{th}-model selector 600-k selects one or more intra-k^{th}-model measurement model candidates from the k^{th} model. The k^{th}-model selector 600-k then determines at least one intra-k^{th}-model measurement model from among the intra-k^{th}-model measurement model candidates. An intra-k^{th}-model measurement model refers to a calculation model for measuring the blood pressure (P) in the blood-pressure measuring unit 160.

In some cases, a pulse wave of predetermined signal quality (high-accuracy pulse wave) cannot be obtained from some of the skin regions. Examples of such skin regions include (i) a skin region partly covered with an object and (ii) a skin region with a shade casted thereon. To improve the accuracy of blood pressure measurement, the fact that there are such skin regions is preferably reflected.

As such, the pulse-wave calculator 17 firstly calculates a pulse wave in each skin region. The pulse-wave calculator 17 may then classify the individual skin regions into (i) a region where a pulse wave of predetermined signal quality has been obtained (hereinafter, a quality-compliant region) and (ii) the other regions (hereinafter, quality-noncompliant regions). A quality-noncompliant region can be also expressed as a region where a pulse wave of predetermined signal quality has not been obtained. For instance, this classification may be performed based on the SNR (an example of signal quality) of each pulse wave. Alternatively, this classification may be performed based on the pixel value of each skin region.

Then, the model selector 60 may extract, as measurement model candidates, only models in which only quality-compliant regions are used, from among the plurality of estimation models. This can more effectively avoid reduction in the accuracy of a measurement result (the blood pressure P, which will be described later on).

Example Process in Body-Movement Classifying Unit 22

N kinds of predetermined patterns regarding the orientation (direction) of the face of the subject H are set in advance in the body-movement classifier 22. Hereinafter, a data set indicating these N kinds of predetermined patterns is referred to as a face-direction template. The body-movement classifier 22 uses the face-direction template to classify a face orientation detected by the body-movement detector 21. That is, the body-movement classifier 22 identifies under which of the patterns within the face-direction template a face direction detected by the body-movement detector 21 (hereinafter, a detected direction) fails.

FIG. 5 is an example face-direction template. In the example of FIG. 5, N=9 is established. The following nine different patterns of face direction are defined in the example of FIG. 5:

Pattern 1, Front;

Pattern 2, Right;

Pattern 3, Upper right;

Pattern 4, Up;

Pattern 5, Upper Left;

Pattern 6, Left;

Pattern 7, Lower Left;

Pattern 8, Down; and

Pattern 9, Lower Right.

The body-movement classifier 22 in this case can classify the detected direction into nine patterns: Patterns 1 to 9. It is noted that Pattern k may be referred to as a k^{th} pattern. The body-movement classifier 22 outputs the classification number (pattern number), which it has identified, every predetermined time.

The foregoing example classification numbers are provided for indicating a transition (change) in the face orientation between two predetermined time points (e.g., Time Points A and B). Let the face orientation at Time Point A fall under Pattern 1. Accordingly, Patterns 1 to 9 can be also expressed as the patterns of the direction of facial movement between Time Points A and B. It is noted that there is no change in the face orientation when the face orientation falls under Pattern 1 even at Time Point B. In this way, these face orientation patterns may be also referred to as patterns of the direction of facial movement.

Reference is made to an instance where the detected direction at Time Point A is front (Pattern 1). In a first example, let the detected direction at Time Point B be left (Pattern 6). The body-movement classifier 22 accordingly changes the classification number to “Pattern 1→Pattern 6” (see the arrow in FIG. 5).

In a second example, let the detected direction at Time Point C (posterior to Time Point B) be front (Pattern 1), and let the detected direction at Time Point D (posterior to Time Point C) be upper right (Pattern 3). The body-movement classifier 22 in this case changes the classification number to “Pattern 1→Pattern 6→Pattern 1→Pattern 3”.

The body-movement classifier 22 can also classify a pattern transition between predetermined two time points. In the second example, the classification of the pattern transition is firstly changed from “Pattern 1→Pattern 6” to “Pattern 6→Pattern 1”. The classification of the pattern transition is then changed from “Pattern 6→Pattern 1” to “Pattern 1→Pattern 3”.

The body-movement classifier 22 can perform pattern classification on the basis of the amount of movement calculated by the body-movement detector 21. For instance, the body-movement classifier 22 may perform pattern classification on the basis of the result of comparison between the amount of movement and a predetermined threshold. Further, a determination process step for pattern classification may use the time average of the amount of movement (hereinafter, the average of the amount of movement) instead of the amount of movement. The time average of the amount of movement may be, for instance, the average of the amount of movement at the time of measuring each measurement data piece for model creation.

Blood-Pressure Measuring Unit 160 and Blood-Pressure-Measurement Outputting Unit 170

The blood-pressure measuring unit 160 uses a measurement model selected by the model selector 60 to measure the blood pressure (P). To be specific, the blood-pressure measuring unit 160 calculates the P by using a pulse wave parameter (e.g., a PTT) calculated by the pulse-wave-parameter calculator 20. In this way, the blood-pressure measuring unit 160 uses the measurement model to calculate the first blood pressure (P) on the basis of the pulse wave parameter.

As described above, the model selector 60 selects a measurement model based on Pattern k (intra-k^{th}-model measurement model). The blood-pressure measuring unit 160 can thus calculate the P by using a measurement model suitable for the face orientation of the subject H.

The blood-pressure-measurement outputting unit 170 acquires the P measured by the blood-pressure measuring unit 160. The blood-pressure-measurement outputting unit 170 then outputs the P as a resultant blood pressure measurement. The blood-pressure-measurement outputting unit 170 may output the P through any notification. For instance, the blood-pressure-measurement outputting unit 170 may be a display. The blood-pressure-measurement outputting unit 170 in this case can visually provide the subject H with the resultant blood pressure measurement by displaying a numeric value indicating the P.

How to Create Measurement Model

FIG. 6 is a flowchart illustrating an example process in the blood-pressure measuring apparatus 1. FIG. 6 illustrates, by way of example, how the blood-pressure measuring apparatus 1 (more specifically, the model setting device 100) creates (sets) a measurement model. This method may be referred to as a method of measurement model creation (or a method of measurement model setting).

The first step is S1, in which the image pickup unit 11 takes a subject image. In S2, the facial-image acquiring unit 14 acquires a facial image (IMG) from the subject image as taken. In S3, the facial-image acquiring unit 14 performs face tracking on the IMG.

The next step is S4, i.e., body movement detection, in which the body-movement detector 21 uses the result of the face tracking in S3 to detect a face orientation (direction of facial movement). In S5, i.e., body movement classification, the body-movement classifier 22 classifies the face orientation as detected in S4. For instance, the body-movement classifier 22 classifies the face orientation into any one (Pattern k) of above Patterns 1 to 9.

The next step is S6, in which the facial-image divider 15 divides the IMG into a plurality of partial regions in accordance with the pattern classified in S5. In S7, the skin-region extractor 16 extracts skin regions from the partial regions. In S8, i.e., pulse wave acquisition, the pulse-wave calculator 17 calculates a pulse wave (pulse wave signal) for each skin region. The next step is S9, i.e., calculation of a pulse wave parameter, in which the pulse-wave-parameter calculator 20 uses the pulse wave to calculate a pulse transit time (PTT) between the skin regions.

Each of the following process steps is performed for each pattern (Pattern k) classified in S5. After S9, S10 is performed, in which the model setting device 100 determines whether there is an estimation model (k^{th} model) based on Pattern k for a current target subject H who undergoes blood pressure measurement. That is, the model setting device 100 determines whether there is an estimation model based on the face orientation. If there is no such estimation model at the moment (If NO in S10), the process proceeds to S11, i.e., measurement of the second blood pressure, in which the blood-pressure acquiring unit 2 acquires the blood pressure (BPm) of the subject H.

Subsequently, the model creating unit 30 (more specifically, the k^{th}-model creating unit 300-k) uses the test data to create a plurality of estimation models (k^{th} models) in Pattern k. To be specific, the model creating unit 30 uses the PTT and BPm to create a plurality of estimation models of predetermined complexity level. This process is S12, i.e., model creation. When S12 is performed for the first time (the first time of this loop process), the model creating unit 30 creates a plurality of estimation models of Complexity Level 1 (a plurality of M1s). As described above, the model creating unit 30 stores each created estimation model (e.g., each M1) in the model storage 50.

The BPm used in S12 is blood pressure that is measured by the blood-pressure acquiring unit 2 at the same time as the image taking of the subject (S1). That is, prior to S11, the process step of measuring the second blood pressure is executed once in advance at the same time as S1.

Subsequently, the evaluation predicted-blood-pressure calculator 41 (more specifically, the k^{th}-model evaluation predicted-blood-pressure calculator 410-k) uses the test data to calculate a predicted blood pressure (BPe, more specifically, BPek) in each M1 created in S12. To be specific, in S13, the evaluation predicted-blood-pressure calculator 41 applies the PTT to each M1 to calculate the BPe.

Subsequently, the model evaluation-index calculator 42 (more specifically, the k^{th}-model evaluation-index calculator 420-k) calculates the evaluation index (PI, more specifically, PIk) of each estimation model. To be specific, in S14, the model evaluation-index calculator 42 calculates the mean square error (MSE) between the BPe and BPm as the P1. As described above, the model evaluation-index calculator 42 stores each calculated PI in the model storage 50. S13 and S14 are also generically referred to as model evaluation. It is noted that prior to S13, the model evaluating unit 40 may read each estimation model (k^{th} model) from the model storage 50.

The next step is S15, in which the model evaluating unit 40 determines whether plotting estimation models having the smallest PI (e.g., MSE) at each complexity level has obtained a minimum MSE. In other words, the model evaluating unit 40 determines whether the smallest MSE at the complexity level calculated in immediately preceding S14 is greater than the smallest MSE at the complexity level calculated in S14 of the last loop process.

When S16 is preformed for the first time (the first time of this loop process), there is no smallest MSE at the complexity level calculated in S14 of the last loop process (i.e., no target to be compared with the smallest MSE at the complexity level calculated in immediately preceding S14). The model evaluating unit 40 thus determines NO in S15 when S15 is performed for the first time.

If no minimum MSE has been obtained (If NO in S15), that is, if the smallest MSE at the complexity level calculated in immediately preceding S14 is smaller than the smallest MSE at the complexity level calculated in S14 of the last loop process, the process proceeds to S16, in which the model creating unit 30 increases the complexity level of the estimation model immediately above. In the foregoing example, the model creating unit 30 increases the complexity level from Level 1 to Level 2. The process then returns to S12. From then on, S12 to S15 are repeated until the model evaluating unit 40 determines YES in S15. Complexity Level z (the maximum complexity level) thus coincides with the number of repeats of S12 to S15.

If a minimum MSE has been obtained (If YES in S15), that is, if the smallest MSE at the complexity level calculated in immediately preceding S14 is greater than the smallest MSE at the complexity level calculated in S14 of the last loop process, the process proceeds to S17, in which the model evaluating unit 40 determines whether the minimum MSE is greater than a predetermined threshold.

If the minimum MSE is greater than the threshold (if YES in S17), the process for creating the k^{th} model ends. This is because that further increase in the complexity level is less likely to offer an estimation model that can provide a better PI (e.g., an MSE) than now. If the minimum MSE is equal to or smaller than the threshold (if NO in S17), the process returns to S16. This is because that further increase in the complexity level is likely to offer an estimation model that can provide a better PI than now.

It is noted that if YES in S10 (if there is already a k^{th} model), the process ends (i.e., S11 to S17 are not performed). This is because that there is no need to create the k^{th} model.

Performing the process in FIG. 6 on each of Patterns 1 to N (e.g., Patterns 1 to 9) enables the model setting device 100 to create the first to N^{th} models (model group).

How to Measure Blood Pressure

FIG. 7 is a flowchart illustrating another example process in the blood-pressure measuring apparatus 1. FIG. 7 illustrates, by way of example, how the blood-pressure measuring apparatus 1 measures blood pressure (i.e., a method of blood pressure measurement). The process steps in FIG. 7 are executed after all the process steps in FIG. 6 complete. That is, before the process steps in FIG. 7 start, (i) the model group created by the model setting device 100 and (ii) the group of evaluation-index sets calculated by the model setting device 100 are stored in the model storage 50 in advance.

Among S21 to S32 in FIG. 7, S21 to S29 are respectively similar to S1 to S9 in FIG. 6. Only process steps relating to S30 to S32 will be thus described. It is noted that the process steps after S30 are performed for each pattern (Pattern k) classified in S25. It is also noted that the measurement of the second blood pressure in the example of FIG. 7 is executed at the same time as S21.

After S29, the model selector 60 (more specifically, the k^{th}-model selector 600-k) reads each estimation model (k^{th} model) from the model storage 50. The model selector 60 also reads each PI from the model storage 50. As described above, the model selector 60 selects a predetermined measurement model (intra-k^{th}-model measurement model) from among a plurality of k^{th} models on the basis of these PIs. In this ways, the model selector 60 selects a predetermined measurement model from among a plurality of estimation models based on face orientations. This process step is S30, i.e., model selection.

The next step is S31, i.e., measurement of the first blood pressure, in which the blood-pressure measuring unit 160 uses the measurement model selected in S30 to measure the blood pressure (P). The final step is S32, in which the blood-pressure-measurement outputting unit 170 outputs the P as a resultant blood pressure measurement. Upon completion of S32, the blood-pressure measuring apparatus 1 finishes blood pressure measurement.

Effects

As earlier described, the measurement instrument in Patent Literature 1 is designed to measure the pulse rate with high accuracy even when the subject H moves his/her body (more specifically, even when the subject H moves his/her face). However, Patent Literature 1 is silent about a specific method for measuring the blood pressure (P) with high accuracy so as to address body movements of the subject H. This conventional technique cannot measure the P with high accuracy.

In contrast, the blood-pressure measuring apparatus 1 includes the model setting device 100, which can set a plurality of kinds of estimation models based on face orientations (that is, based on body movements). The blood-pressure measuring apparatus 1 can also use these estimation models, stored in the model storage 50, to measure (calculate) the blood pressure (P). That is, the blood-pressure measuring apparatus 1 can measure the by reflecting body movements of the subject H. This can measure the P more accurately than before even when a user moves his/her body.

### Second Embodiment

(1) A subject H is not limited to a human. The subject H needs to be a target to which the blood-pressure measuring method according to one aspect of the present disclosure is applicable. For instance, the subject H may be an animal, including a dog and a cat.

(2) An ROI is not limited to a face. The ROI needs to be the body surface of a living body through which its pulse wave can be obtained. Other examples of the ROI include a neck, a chest, and a palm.

However, the ROI is preferably a face. Using an IMG (facial image) can reduce a burden on the subject H during blood pressure measurement. That is, using an IMG facilitates measuring the blood pressure of the subject H who is under natural conditions (or is relaxed).

(3) The body-movement detector 21 does not necessarily have to detect a body movement on the basis of an image analysis (e.g., using the result of face tracking). For instance, the body-movement detector 21 may be a contact sensor capable of detecting a body movement. This sensor needs to be designed not to make the subject H feel restrained as much as possible. As described above, the blood-pressure measuring apparatus 1 may be a contact blood-pressure measuring apparatus.

The body-movement detector 21 may further detect at least one of (i) the velocity of a body movement and (ii) the acceleration of the body movement in addition to the degree (the amount of displacement) of the body movement. The body-movement classifier 22 in this case may classify the pattern of the body movement on the basis of the degree of the body movement and further on the basis of at least one of (i) the velocity of the body movement and (ii) the acceleration of the body movement. The model creating unit 30 in this case needs to create an estimation model based on this classification.

(4) A pulse wave parameter is not limited to a PTT. For instance, the pulse-wave-parameter calculator 20 may calculate the amount of waveform characteristics of a pulse wave in each skin region as a waveform parameter. Examples of the amount of waveform characteristics usable include (i) pulse wave oscillation and (ii) a time difference between pulse wave pulses. The pulse-wave-parameter calculator 20 may also combine a PTT and the amount of waveform characteristics together to set a pulse wave parameter.

(5) An estimation model is not limited to a linear model. The model creating unit 30 may create a non-linear model (a calculation model expressed by a non-linear function) through regression analysis.

(6) The evaluation index (PI) of an estimation model is not limited to the mean square error between BPe and BPm. For instance, (i) the mean absolute error between the BPe and BPm or (ii) the standard deviation of error between the BPe and BPm can be used as the PI. Furthermore, the PI needs to be a parameter calculated based on the BPe and BPm and is not limited to an error-related parameter. For instance, (i) adjusted index of determination and (ii) Akaike's information criteria (AIC) can be used as the PI.

(7) The model storage 50 needs to be communicably connected to the blood-pressure measuring apparatus 1. For instance, the model storage 50 may be a server external to the blood-pressure measuring apparatus 1. The model storage 50 thus does not necessarily have to be disposed within the blood-pressure measuring apparatus 1. Likewise, the model storage 50 does not necessarily have to be disposed within the model setting device 100.

The model storage 50 can be omitted. The model selector 60 in this case needs to acquire each measurement model directly from the model creating unit 30. Likewise, the model selector 60 needs to acquire each P1 directly from the model evaluating unit 40. However, the model storage 50 is preferably provided in order for the blood-pressure measuring apparatus 1 to speedily measure the P.

(8) The model setting device 100 may further include a measurement-data classifier. The measurement-data classifier is a functional unit that (i) extracts a pulse wave for each of multiple data measuring times from the pulse waves acquired by the pulse-wave acquiring unit 10, and that (ii) classifies each extracted pulse wave. For instance, the model creating unit 30 may serve as such a measurement-data classifier.

The multiple data measuring times include, for instance, four different times: 5 seconds, 10 seconds, 20 seconds, and 30 seconds. The pulse-wave-parameter calculator 20 uses each pulse wave classified by the measurement-data classifier, to calculate a plurality of pulse wave parameters. That is, the pulse-wave-parameter calculator 20 calculate these pulse wave parameters (i) for each pattern classification made by the body-movement classifier 22 and (ii) for each pulse wave classification made by the measurement-data classifier.

In this example, the pulse-wave-parameter calculator 20 calculates four different pulse wave parameters in Pattern k: a pulse wave parameter at a data measuring time of 5 seconds, a pulse wave parameter at a data measuring time of 10 seconds, a pulse wave parameter at a data measuring time of 20 seconds, and a pulse wave parameter at a data measuring time of 30 seconds.

The model creating unit 30 then calculates each estimation model by using each pulse wave parameter calculated by the pulse-wave-parameter calculator 20. This configuration enables estimation model creation for each data measuring time. The blood-pressure measuring apparatus 1 can hence select a measurement model suitable for blood pressure measurement by further reflecting the time duration of a body movement. This can further improve the accuracy of blood pressure measurement.

(9) The model setting device 100 needs to be communicably connected to the blood-pressure measuring apparatus 1. For instance, the model setting device 100 may be a server external to the blood-pressure measuring apparatus 1. The model setting device 100 thus does not necessarily have to be disposed within the blood-pressure measuring apparatus 1.

### Third Embodiment

The control blocks of the blood-pressure measuring apparatus 1 (in particular, the pulse-wave acquiring unit 10, the pulse-wave-parameter calculator 20, the body-movement detector 21, the body-movement classifier 22, the model creating unit 30, the model evaluating unit 40, the model selector 60, and the blood-pressure measuring unit 160) may be implemented by a logic circuit (hardware) formed in, for instance, an integrated circuit (IC chip) or by software.

For software, the blood-pressure measuring apparatus 1 includes a computer that executes commands of a program, which is software that implements each function. The computer includes, for instance, at least one processor (controller) and at least one computer-readable recording medium storing the program. The processor in the computer reads the program from the recording medium and executes the program, thus achieving the object of one aspect of the present disclosure. An example of the processor usable is a central processing unit (CPU). An example of the recording medium usable is a non-transitory tangible medium, including a read-only memory (ROM), a tape, a disc, a card, a semiconductor memory, and a programmable logic circuit. The computer may also include, for instance, a random access memory (RAM) that develops the program. The program may be supplied to the computer via any transmission medium (e.g., a communication network or a broadcast wave) capable of transmitting the program. The blood-pressure measuring apparatus 1 may be implemented by a publicly known information processor (e.g., a smartphone, a tablet, or a personal computer). One aspect of the present disclosure can be implemented also in the form of a data signal in which the program is embodied through electronic transmission and that is embedded in a carrier wave.

Additional Remarks

The present disclosure is not limited to the foregoing embodiments. Various modifications can be devised within the scope of the claims. In addition, an embodiment that is obtained in combination, as appropriate, with the technical means disclosed in the respective embodiments is also included in the technical scope of one aspect of the present disclosure. Furthermore, combining the technical means disclosed in the respective embodiments can offer a new technical feature.

1-6. (canceled)

7. A blood-pressure measuring apparatus that measures a first blood pressure of a living body on the basis of a pulse wave of the living body, the blood-pressure measuring apparatus comprising:
a pulse-wave acquiring unit configured to acquire the pulse wave in a predetermined region on a body surface of the living body;
a body-movement detector configured to detect a direction of movement of the predetermined region;
a body-movement classifier configured to classify the direction of movement; and
a pulse-wave-parameter calculator configured to calculate a plurality of pulse wave parameters based on the pulse wave,
wherein the blood-pressure measuring apparatus is communicably connected to a model storage,
the model storage stores in advance a blood-pressure estimation model for estimating the first blood pressure in response to a resultant classification of the direction of movement, and
the blood-pressure measuring apparatus further comprises a first-blood-pressure measuring unit configured to calculate, by using the blood-pressure estimation model, the first blood pressure based on the plurality of pulse wave parameters.

8. The blood-pressure measuring apparatus according to claim 7, wherein
the model storage stores in advance
a plurality of blood-pressure estimation models for estimating the first blood pressure in response to the resultant classification of the direction of movement, and
resultant evaluations of the plurality of individual blood-pressure estimation models based on the resultant classification of the direction of movement, and
the blood-pressure measuring apparatus further comprises:
a model selector configured to select a measurement model for calculating the first blood pressure from among the plurality of blood-pressure estimation models in response to the resultant evaluations of the plurality of individual blood-pressure estimation models; and
a first-blood-pressure measuring unit configured to calculate, by using the measurement model, the first blood pressure based on the plurality of pulse wave parameters.

9. The blood-pressure measuring apparatus according to claim 8, wherein
the body-movement classifier classifies the direction of movement into N different patterns including first to N^{th} patterns, where N is an integer equal to or greater than two,
the plurality of blood-pressure estimation models include a blood-pressure estimation model that is based on a k^{th} pattern and is defined as a k^{th} model, where k is an integer ranging from one to N inclusive,
the model storage stores in advance
(i) a plurality of k^{th} models that are the plurality of blood-pressure estimation models based on the k^{th} pattern, and
(ii) resultant evaluations of the plurality of individual k^{th} models, and
the model selector has a k^{th}-model selector configured to select the measurement model from among the plurality of k^{th} models in response to the resultant evaluations of the plurality of individual k^{th} models.

10. The blood-pressure measuring apparatus according to claim 7, wherein
the predetermined region is a face of the living body.

11. The blood-pressure measuring apparatus according to claim 8, wherein
the predetermined region is a face of the living body.

12. The blood-pressure measuring apparatus according to claim 9, wherein
the predetermined region is a face of the living body.

13. A model setting device communicably connected to a blood-pressure measuring apparatus that measures a first blood pressure of a living body on the basis of a pulse wave of the living body, the model setting device comprising:
a second-blood-pressure measuring unit configured to measure a second blood pressure of the living body;
a pulse-wave acquiring unit configured to acquire the pulse wave in a predetermined region on a body surface of the living body;
a body-movement detector configured to detect a direction of movement of the predetermined region;
a body-movement classifier configured to classify the direction of movement; and
a pulse-wave-parameter calculator configured to calculate a plurality of pulse wave parameters based on the pulse wave,
wherein the model setting device is communicably connected to a model storage, and
the model setting device further comprises a model creating unit configured to create, based on the plurality of pulse wave parameters and the second blood pressure, a blood-pressure estimation model for estimating the first blood pressure in response to a resultant classification of the direction of movement, the model creating unit being configured to store the blood-pressure estimation model in the model storage.

14. The model setting device according to claiml3, further comprises
a model evaluating unit configured to individually evaluate the plurality of blood-pressure estimation models stored in the model storage in response to the resultant classification of the direction of movement, the model evaluating unit being configured to store resultant evaluations of the plurality of individual blood-pressure estimation models in the model storage.

15. The model setting device according to claim 14, wherein
the body-movement classifier classifies the direction of movement into N different patterns including first to N^{th} patterns, where N is an integer equal to or greater than two,
the plurality of blood-pressure estimation models include a blood-pressure estimation model that is based on a k^{th} pattern and is defined as a k^{th} model, where k is an integer ranging from one to N inclusive,
the model creating unit has a k^{th}-model creating unit configured to create, based on the plurality of pulse wave parameters and the second blood pressure, a plurality of k^{th} models for estimating the first blood pressure, the k^{th}-model creating unit being configured to store the plurality of k^{th} models in the model storage, and
the model evaluating unit has a k^{th}-model evaluating unit configured to individually evaluate the plurality of k^{th} models stored in the model storage, the k^{th}-model evaluating unit being configured to store resultant evaluations of the plurality of individual k^{th} models in the model storage.

16. A blood-pressure measuring method using a blood-pressure measuring apparatus that measures a first blood pressure of a living body on the basis of a pulse wave of the living body, the blood-pressure measuring method comprising the steps of:
acquiring the pulse wave in a predetermined region on a body surface of the living body;
detecting a direction of movement of the predetermined region;
classifying the direction of movement; and
calculating a plurality of pulse wave parameters based on the pulse wave,
wherein the blood-pressure measuring apparatus is communicably connected to a model storage,
the model storage stores in advance a blood-pressure estimation model for estimating the first blood pressure in response to a resultant classification of the direction of movement, and
the blood-pressure measuring method further comprises the step of calculating, by using the blood-pressure estimation model, the first blood pressure based on the plurality of pulse wave parameters.

17. The blood-pressure measuring method according to claim 16, wherein
the model storage stores in advance
a plurality of blood-pressure estimation models for estimating the first blood pressure in response to the resultant classification of the direction of movement, and
resultant evaluations of the plurality of individual blood-pressure estimation models based on the resultant classification of the direction of movement, and
the blood-pressure measuring method further comprises the steps of:
selecting a measurement model for calculating the first blood pressure from among the plurality of blood-pressure estimation models in response to the resultant evaluations of the plurality of individual blood-pressure estimation models; and
calculating, by using the measurement model, the first blood pressure based on the plurality of pulse wave parameters.
